(19) United States
(12) Patent Application Publication
(10) Pub. No.: US 2007/0134689 A1
(43) Pub. Date: Jun. 14, 2007

(54) Tumor marker for ovarian cancer diagnosis

(75) Inventor: Song-Nan Chow, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: National Taiwan University, Taipei (TW)

(21) Appl. No.: 11/532,079

(22) Filed: Sep. 14, 2006

Related U.S. Application Data
(60) Provisional application No. 60/721,840, filed on Sep. 28, 2005.

(51) Int. Cl. C12Q 1/68; G01N 33/574

(52) U.S. Cl. 435/6; 435/7.23

(57) ABSTRACT

The present invention relates to a tumor marker for diagnosis of ovarian cancer, which is selected from the group consisting of alectin-1, cathepsin B, MHC class I antigen, heat shock protein (HSP) 27, ubiquitin carboxy-termal esterase L1, cellular retinol-binding protein (CRBP), transthyretin, SH3 binding glutamate-rich protein, tubulin-specific chaperone A, RNA binding protein regulatory subunit, γ-actin, tropomyosin and calcium/calmodulin-stimulated cyclic nucleotide phosphatase. The ovarian cancer is diagnosed effectively and efficiently based on detecting the expression levels of the tumor markers in the invention from the ovarian tissue sample of an individual to be diagnosed.

### BACKGROUND OF THE INVENTION

1. Field of the Invention

The present invention relates to a tumor marker for diagnosis of cancer, especially for diagnosis of ovarian cancer, which can be applied in early diagnosis of ovarian cancer.

2. The Prior Arts

Human ovarian cancer is one of the common gynecological malignancies. In the developed country, it is one of the leading causes of death of the gynecological cancers, and the five-year survival rate is only about 30%. Overall about one woman in 70 will get ovarian cancer, and estimated one woman in 100 will die from this cancer in USA. This is because the illness is often diagnosed during late stage of the cancer. The cancer has often spread beyond the ovaries at that time, and therefore related to the low survival rate. Though Taiwanese women do not have a high incidence of ovarian cancer, but the incidence has increased over past two decades.

The five-year survival rate will up to 90-95 percent if the ovarian cancer is caught very early according to previous medical reports. The lack of reliable tumor marker has made the early detection of ovarian cancer difficult. Therefore most of the ovarian cancer patients will be diagnosed when the cancer cells have been spread. The survival rate thus cannot be lowered.

CA-125, cancer antigen-125, is a protein that may be released into the bloodstream, and is found at levels in most ovarian cancer cells. It is also a serum marker being studied thoroughly. The known detection method for ovarian cancer is the measurement of CA-125 in serum to assess the risk of having ovarian cancer.

The CA-125 test only returns a true positive result for about 50% of Stage I ovarian cancer patients though it has an 80% chance of returning true positive results from stage II, III, and IV ovarian cancer patients. It yields many false positive results. Therefore it is not recommended as a diagnostic tool or target for ovarian cancer in early stage cancers. Due to the current limitation to early diagnosis of ovarian cancer, it is important to search and identify new potential biomarkers in ovarian cancer.

### SUMMARY OF THE INVENTION

In order to overcome the drawbacks of the prior art as described above, a primary object of the present invention is to provide an ovarian cancer marker to properly detect ovarian cancer at an early stage.

Another object of the present invention is to provide a method for the detection of ovarian cancer to identify ovarian cancer at an early stage.

To fulfill the objective of the present invention, a tumor marker for ovarian cancer diagnosis is selected from the group consisting of galectin-1, cathepsin B, MHC class I antigen, heat shock protein 27 (HSP 27), ubiquitin carboxy-termal esterase L1, cellular retinol-binding protein (CRBP), transthyretin, SH3 binding glutamate-rich protein, tubulin-specific chaperone A, RNA binding protein regulatory subunit, γ-actin, tropomyosin and calcium/calmodulin-stimulated cyclic nucleotide phosphatase.

Compared with normal ovarian tissues, the cancer marker of the present invention is either up-regulated or down-regulated in ovarian tissues from ovarian cancer patients.

In addition, a method for detecting ovarian cancer according to the present invention comprises the steps of:
(1) obtaining an ovarian tissue sample from an individual to be diagnosed;
(2) determining the expression levels of the abovementioned tumor markers in the ovarian tissue sample;
(3) comparing the expression levels of the tumor markers in the ovarian tissue sample of step (2) with the expression levels of the tumor markers in non-cancerous ovarian tissues; and
(4) identifying if the individual being diagnosed is affected with the ovarian cancer or not from result of step (3).

The tumor markers according to the present invention can be applied as a diagnostic tool in detecting ovarian cancer at an early stage. In addition, the detection method for the diagnosis of ovarian cancer according to the present invention can also be applied in detecting ovarian cancer at an early stage.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

FIG. 1 shows Western blot analysis of cathepsin B, galectin-1, RNA binding protein regulatory subunit, and cellular retinol-binding protein (CRBP) from normal ovarian tissue and ovarian cancer tissue samples.

### DETAILED DESCRIPTION OF THE PREFERRED EMBODIMENT

The tumor marker of the present invention can be applied in early diagnosis of ovarian cancer. Compared with normal ovarian tissues, the expression level of tumor marker according to the present invention is either up-regulated or down-regulated in ovarian tissue of ovarian cancer patient. Following comparison between tumor tissues and corresponding normal tissues, the examples for up-regulated tumor marker are galectin-1, cathepsin B, MHC class I antigen, heat shock protein (HSP) 27 and ubiquitin carboxy-termal esterase L1. On the contrary, the examples for down-regulated tumor marker are cellular retinol-binding protein (CRBP), transthyretin, SH3 binding glutamate-rich protein, tubulin-specific chaperone A, RNA binding protein regulatory subunit, γ-actin, tropomyosin and calcium/calmodulin-stimulated cyclic nucleotide phosphatase.

The changes of expression level of the tumor marker in the ovarian tissues according to the present invention can be easily determined with the relevant known protein analysis techniques, which include but are not limited to polyacrylamide gel electrophoresis (PAGE), Western blot, Dot blot and so on, by the person skilled in the art after reading the disclosure of the specification. A Example for polyacrylamide gel electrophoresis include but is not limited to sodium dodecyl sulfate polyacrylamide gel electrophoresis (SDS-PAGE).

When the expression levels of the tumor markers in the ovarian tissues according to the present invention were analyzed with PAGE combined with ImageMaster™ 2D Elite, the preferred ratio of signal strength for up-regulated protein dots (tumor markers) in the gel slabs after gel electrophoresis is larger or equal to 1.25 (ovarian cancer samples in comparison with normal ovarian tissues); and the preferred ratio of signal strength for down-regulated protein dot (tumor markers) in the gel slabs is smaller or equal to 0.8 (ovarian cancer samples in comparison with normal ovarian tissues). The gel slabs after gel electrophoresis were stained with Silver staining solution or Coomassie Brillant Blue staining solution.

The present invention also provides a method for the detection of ovarian cancer to identify ovarian cancer at an early stage since the expression levels of the tumor markers in the ovarian tissues will be changed in accordance with the invasion of tumor cells. The method for detecting ovarian cancer according to the invention first obtains an ovarian tissue sample from an individual to be diagnosed; and then analyzes the expression levels of the tumor markers of the present invention in the ovarian tissue sample through the known methods. Then the expression levels of the tumor markers in the ovarian tissue sample are compared with the expression levels of the tumor markers in normal ovarian tissues. Lastly, comparison result of the expression levels of tumor markers is used to determine whether the expression level has been changed (up-regulated or down-regulated), and to identify if the individual being diagnosed is affected with the ovarian cancer or not.

The abovementioned normal ovarian tissues could be obtained from other individual, who is not being affected by the ovarian cancer; or other parts of the ovarian tissue sample to be diagnosed, which is not invaded by the cancer, in the same individual.

The abovementioned method to analyze the expression levels of the tumor markers can be easily performed with the relevant known protein analysis techniques, which include but are not limited to polyacrylamide gel electrophoresis (PAGE), Western blot, Dot blot and so on, by the person skilled in the art after reading the disclosure of the specification. Examples for polyacrylamide gel electrophoresis include but are not limited to sodium dodecyl sulfate polyacrylamide gel electrophoresis (SDS-PAGE).

The abovementioned expression level changes of the tumor markers can be up-regulated or down-regulated. For example, PAGE combined with ImageMaster™ 2D Elite is used to analyze the expression levels of the tumor markers in the ovarian tissues according to the present invention. When the ratio of signal strength for up-regulated protein dots (tumor markers) in the gel slabs after gel electrophoresis is larger or equal to 1.25 (ovarian cancer samples in comparison with normal ovarian tissues); or the ratio of signal strength for down-regulated protein dot (tumor markers) in the gel slabs is smaller or equal to 0.8 (ovarian cancer samples in comparison with normal ovarian tissues), the individual is identified to be affected with ovarian cancer. On the contrary, the individual is identified not to be affected with ovarian cancer if the signal ratio is within the abovementioned value range.

The abovementioned ovarian cancer comprises clinical stage I, II, III, and IV ovarian cancer.

In addition, the accuracy of diagnosis of ovarian cancers performed with the method of the present invention can be increased through combining the analysis results of the expression levels from multiple of the tumor markers.

### EXAMPLE 1

### Screening of Tumor Markers

The ovarian tissues collected in the present invention comprised of 36 epithelial ovarian cancers, 10 borderline malignancies and 18 normal ovaries. Clinical and histological characteristics of these 36 ovarian cancer tissue samples are summarized in Table 1.

The histologic subtypes of ovarian cancer include clear cell, endometrioid, mucinous, serous and others as shown in Table 1. Among these 36 ovarian cancer tissue samples, 10 were of clinical stage I, 6 of clinical stage II, 18 of clinical stage III, and 2 of clinical stage IV.

Protein extracts from the normal ovarian tissues and the ovarian cancer tissues were separated on SDS-PAGE followed by 2D-polyacrylamide gel electrphoresis.

2D-polyacrylamide gel electrophoresis was performed on a 130 mm, linear immobilized pH 4-7 Immobiline DryStrip (Amersham Pharmacia Biotech, Piscataway, N.J., USA) using Multiphor II Electrophoresis system. The ovarian tissues were frozen in liquid nitrogen and grinded to a fine powder. The powder was extracted with an extract buffer containing phosphate buffered saline (PBS) buffer and protease inhibitor. The supernatant was precipitated with trichloroacetic acid (TCA) to final concentration of 5% after extraction solution was centrifuged. The precipitated pellet was resuspended in buffer containing 8 M urea and 0.1 M dithiothreitol (DTT).

Portions of 450 μg of samples were rehydrated overnight at room temperature. After rehydration, the gel electrophoresis was carried out at 400 V for 1 h, followed by a linear gradient from 400 V to 3500 V for 1.5 h, and fixed at 3500 V for a total of 70 kVh. Prior to the second-dimension separation, the Immobiline DryStrips were pre-equilibrated with equilibration buffer containing 0.05 M Tris-HCl (pH 8.8), 6 M urea, 2% (w/v) sodium dodecyl sulfate (SDS), 30% (v/v) glycerol, and 1% (w/v) dithiothreitol for 15 min. Then the strips were re-equilibrated in the same equilibration buffer but replacing dithiothreitol with 2.5% (w/v) iodoacetamide for 15 min. For the second-dimension separation, the serum proteins were separated in a 12.5% polyacrylamide gel in running buffer containing 0.025 M Tris pH 8.8, 0.192 M Glycine and 0.1% SDS. The second dimension gels were electrophoresed at constant current 10 mA through the stacking gel and at 20 mA through the separating gel.

The gel slabs were stained by the Silver stain method or with Coomassie Blue solution containing 0.25% (w/v) Coomassie Brilliant Blue R 250, 35% (v/v) methanol, and 7% (v/v) acetic acid. Then each gel was de-stained with 35% methanol/7% acetic acid.

Protein spots in gel slabs were different in intensity after staining. Proteins in high levels generate deep color (high intensity), while low levels generate light color (low intensity). The protein band intensities of the cancer tissue and normal tissues in the gel slabs were compared. And 13 protein spots were selected from 2D gels as representative protein spots among normal ovaries, borderline serous ovarian tumors and invasive ovarian carcinomas. Selected protein spots from 2D gels were excised, double distilled water (ddH₂O) washed, and destained with 0.025 M ammonium bicarbonate/50% acetonitrile (ACN). The protein in the protein spot was digested overnight with trypsin at 37° C., and the proteolytic peptide fragments were extracted with 1% Trifluoroacetic acid (TFA)/50% ACN. After lyophilized, the extracted peptides were dissolved in 30% ACN and mixed with matrix solution, then subjected to matrix assisted laser desorption ionization time-of-flight mass spectrometry (MALDI-TOF MS) analysis.

MALDI mass spectra were obtained using an Autoflex workstation (Bruker-Daltonics, Bremen, Germany) equipped with a 337-nm wavelength nitrogen laser. The peptide spectra, acquired in reflection mode at an accelerating voltage of 20 kV, were the sum of 50 laser shots. The mass spectra were externally calibrated using low mass peptide standards. This procedure typically results in mass accuracies of 50-100 ppm. The de-isotope tryptic peptide fragments were used for protein identification by using the MASCOT search engine (http://www.matraxscience.com) based on the peptide mass fingerprinting of entire NCBI and SwissPort protein databases.

Thirteen protein spots identified through entire NCBI and SwissPort protein databases, were galectin-1, cathepsin B, MHC class □antigen, heat shock protein 27 (HSP 27), ubiquitin carboxy-termal esterase L 1, cellular retinol-binding protein (CRBP), transthyretin, SH3 binding glutamate-rich protein, tubulin-specific chaperone A, RNA binding protein regulatory subunit, γ-actin, tropomyosin and calcium/calmodulin-stimulated cyclic nucleotide phosphatase. Characterization of these 13 protein spots was listed in Table 2.

On the other hand, signal strengths of the Silver-stained gel slabs were detected with ImageMaster™ 2D Elite software (Amersham Biosciences biotech, NJ, USA) to analyze the differential expression of protein spots in 2D-gal electrophoresis among normal, borderline and malignant ovarian tissues, and the results were shown in Table 3.

ImageMaster™ 2D Elite result from Table 3 showed that galectin-1, cathepsin B, MHC class I antigen, HSP 27 and ubiquitin carboxy-termal esterase L1 were up-regulated (signal strength ≧1.25 as compared with normal ovarian tissue) in ovarian cancer tissues; while CRBP, transthyretin, SH3 binding glutamate-rich protein, tubulin-specific chaperone A, RNA binding protein regulatory subunit, γ-actin, tropomyosin and calcium/calmodulin-stimulated cyclic nucleotide phosphatase were down-regulated (signal strength ≦0.80 as compared with normal tissue).

### EXAMPLE 2

Four of the protein spots detected in 2D-gel electrophoresis images, which included: cathepsin B, galectin-1, RNA-binding protein regulatory subunit, and Cellular Retinol-binding protein (CRBP), were further identified through Dot blot, or SDS-PAGE followed by Western blotting analyses.

The tissues from normal or cancer ovaries were grinded with plastic pestles in ¼ PBS buffer containing protease inhibitor cocktail (Calbiochem). After centrifugation 15,000×g for 10 min at 4□, the supernatant was transferred to an eppendorf tube and subjected to Dot blot, or SDS-PAGE followed by Western blotting analyses. The protein concentration was determined by the absorption of A₂₈₀.

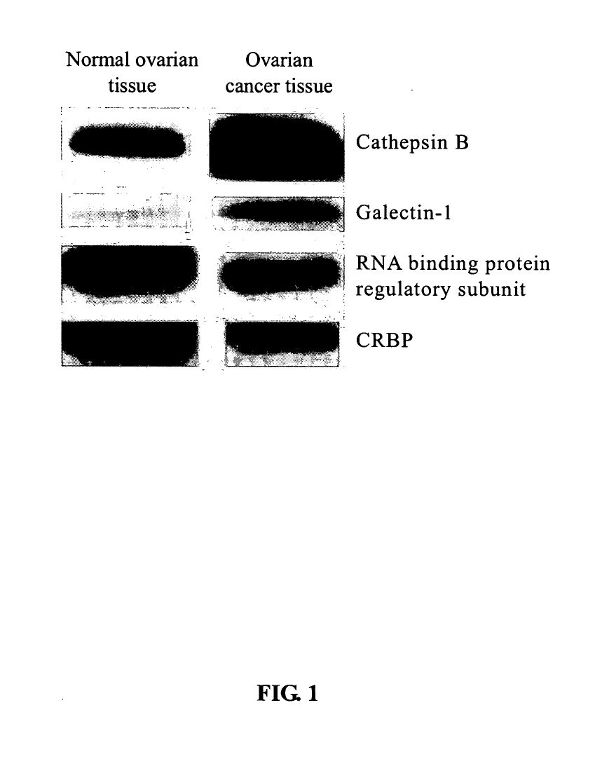

For SDS-PAGE analysis, 30 μg of protein sample was applied to each lane. All samples were heated for 5 min at 95□ before loading into the 15% polyacrylamide gel. After electrophoresis, proteins were electroblotted onto polyvinylidene difluoride (PVDF) membrane. For Dot blot analysis, 5 μg of protein was loaded onto PVDF membrane directly. The membranes were blocked in a blocking solution (5% nonfat dried milk in 1×PBS with 2% Tween-20) for 1 hr at room temperature. The membranes were then probed with anti-retinol binding protein antibody (USBiological, Cat# R1701-16), anti-cathepsin B antibody (USBiological, Cat# C2097-03D), anti-PARK7 antibody (USBiological, Cat# P3111), and anti-galectin-1 antibody (Novocastra, Cat# NCL-GAL1) in blocking solution for 2 hr at room temperature. After washing with the PBST solution (0.05% Tween-20 in 1×PBS), the membranes were incubated with horseradish peroxidase-conjugated anti-immunoglobulin antibody in the blocking solution for 1 hr at room temperature. After additional wash with the PBST solution, membranes were developed with Western Lightning Chemiluminescence Reagent Plus (PerkinElmer). The membranes were scanned using an UMAX Astra 4000U scanner (http://www.umax.com/world/) to detect the signals. The signal strengths from Dot blot were quantified with a GenePix 6.0 software (http://www.moleculardevices.com/) (Table 4), and the images from Western blotting were analyzed with a Fujifilm Science Lab 98 software (Image Gauge V3.12) (FIG. 1).

Results from Table 4 and FIG. 1 showed that both cathepsin B and galectin-1 were up-regulated, while RNA binding protein regulatory subunit and cellular retinol-binding protein (CRBP) were down-regulated in the ovarian cancer tissue samples. These results are in accordance with Example 1.

TABLE 1
Clinical and histologic characteristics
of ovarian cancer tissue samples.

| | | | | Grade of |
|---|---|---|---|---|
| | | | | Differen- |
| No. | Age | Histologic type | Stage | tiation* |
| 1 | 43 | Clear cell carcinoma | Ia | III |
| 2 | 45 | Clear cell carcinoma | Ia | III |
| 3 | 48 | Clear cell carcinoma | Ia | III |
| 4 | 43 | Clear cell carcinoma | Ib | III |
| 5 | 52 | Clear cell carcinoma | Ic | III |
| 6 | 48 | Endometrioid adenocarcinoma | Ia | I |
| 7 | 81 | Endometrioid adenocarcinoma | Ia | I |
| 8 | 46 | Endometrioid adenocarcinoma | Ic | I |
| 9 | 48 | Mucinous cystadenocarcinoma | Ia | I |
| 10 | 36 | Mucinous cystadenocarcinoma | Ia | II |
| 11 | 65 | Serous papillary adenoarcinoma | II | II |
| 12 | 41 | Serous cyadenocarcinoma | IIa | III |
| 13 | 56 | Serous adenocarcinoma | IIa | III |
| 14 | 56 | Serous carcinoma | IIa | II |
| 15 | 70 | Serous cystadenocarcinoma | IIb | II |
| 16 | 59 | Endometrioid adenocarcinoma | IIa | I |
| 17 | 62 | Serous cystadenocarcinoma | III | III |
| 18 | 54 | Serous papillary adenoarcinoma | III | III |
| 19 | 61 | Squamous cell carcinoma | IIIa | III |
| 20 | 58 | Clear cell carcinoma | IIIc | III |
| 21 | 60 | Clear cell carcinoma | IIIc | III |
| 22 | 46 | Endometrioid adenocarcinoma | IIIb | I |
| 23 | 44 | Endometrioid adenocarcinoma | IIIc | III |
| 24 | 56 | Serous carcinoma | IIIb | III |
| 25 | 61 | Serous cystadenocarcinoma | IIIc | II |
| 26 | 70 | Serous papillary adenoarcinoma | IIIb | II |
| 27 | 78 | Serous papillary adenoarcinoma | IIIb | III |
| 28 | 42 | Serous papillary adenoarcinoma | IIIc | II |
| 29 | 70 | Serous papillary adenoarcinoma | IIIc | II |
| 30 | 46 | Serous papillary adenoarcinoma | IIIc | II |
| 31 | 82 | Serous papillary adenoarcinoma | IIIc | III |
| 32 | 72 | Serous papillary adenoarcinoma | IIIc | III |
| 33 | 71 | Serous surface papillary adeno- | IIIb | II |
| | | carcinoma | | |
| 34 | 59 | Serous surface papillary adeno- | IIIc | III |
| | | carcinoma | | |
| 35 | 47 | Mixed adenocarcinoma (Endometri- | IV | III |
| | | oid & Serous) | | |
| 36 | 51 | Serous cystadenocarcinoma | IV | III |

*Differentiation: Grade I, well differentiation; Grade II, moderate differentiation; Grade III, poor differentiation.

TABLE 2
List of protein spots demonostrating differential
expression in 2D-gel electrophoresis among different
types of ovarian tissues in this study

| | | Acession | | |
|---|---|---|---|---|
| No. | Protein Name | Number | PI | M.W. |
| 1 | Galectin-1 | P09382 | 5.33 | 14715.70 |
| 2 | Cathepsin B | 2007265A | 5.44 | 17154.04 |
| 3 | MHC class I antigen | CAI40345 | 6.33 | 21230.37 |
| 4 | HSP 27 | BAB17232 | 5.98 | 22782.52 |
| 5 | Ubiquitin carboxyl-terminal | NP_004172 | 5.33 | 24824.34 |
| | esterase L1 | | | |
| 6 | Cellular Retinol-binding protein | AAA31113 | 5.41 | 23038.88 |
| | (CRBP) | | | |
| 7 | Transthyretin | AAA49620 | 5.41 | 16445.58 |
| 8 | SH3 binding glutamate-rich | JE0178 | 5.22 | 12774.25 |
| | protein | | | |
| 9 | Tubulin-specific chaperone A | AAP36018 | 5.25 | 12854.83 |
| 10 | RNA-binding protein regulatory | AAH08188 | 6.33 | 19891.05 |
| | subunit | | | |
| 11 | γ-Actin | AAB59376 | 5.23 | 42051.03 |
| 12 | Tropomyosin | AAB59509 | 4.63 | 32989.81 |
| 13 | Calcium/calmodulin- stimulated | AAB50018 | 6.07 | 21246.42 |
| | cyclic nucleotide phosphatase | | | |

TABLE 3
Analysis of signal strengths of differential expression
of protein spots in 2D-gel electrophoresis maps among
normal, borderline and malignant ovarian tissue.

| Protein | | Types of tissue samples | | |
|---|---|---|---|---|
| spot | | Normal | Borderline | Malignant |
| number | Protein names | (n = 18) | (n = 10) | (n = 36) |
| 1 | Galectin-1 | 1.00 ± 0.54 | 0.49 ± 0.29 | 1.34 ± 1.01 |
| 2 | Cathepsin B | 1.00 ± 0.20 | 0.60 ± 0.54 | 1.43 ± 1.09 |
| 3 | MHC class I antigen | 1.00 ± 0.30 | 1.17 ± 0.57 | 1.48 ± 0.85 |
| 4 | HSP 27 | 1.00 ± 0.35 | 1.25 ± 0.50 | 1.31 ± 0.66 |
| 5 | Ubiquitin carboxyl- | 1.00 ± 0.33 | 1.49 ± 0.54 | 1.27 ± 0.44 |
| | terminal esterase L1 | | | |
| 6 | Cellular Retinol- | 1.00 ± 0.56 | 0.51 ± 0.87 | 0.25 ± 0.35 |
| | binding protein | | | |
| | (CRBP) | | | |
| 7 | Transthyretin | 1.00 ± 0.70 | 0.86 ± 0.78 | 0.74 ± 0.76 |
| 8 | SH3 binding | 1.00 ± 0.72 | 0.42 ± 0.45 | 0.62 ± 0.54 |
| | glutamate-rich | | | |
| | protein | | | |
| 9 | Tubulin-specific | 1.00 ± 0.41 | 1.25 ± 1.68 | 0.68 ± 1.08 |
| | chaperone A | | | |
| 10 | RNA-binding | 1.00 ± 0.75 | 0.57 ± 0.43 | 0.74 ± 0.61 |
| | protein | | | |
| | regulatory | | | |
| | subunit | | | |
| 11 | γ-Actin | 1.00 ± 0.33 | 0.21 ± 0.11 | 0.15 ± 0.09 |
| 12 | Tropomyosin | 1.00 ± 0.30 | 0.89 ± 0.41 | 0.78 ± 0.38 |
| 13 | Calcium/calmodu- | 1.00 ± 0.31 | 0.88 ± 0.36 | 0.75 ± 0.29 |
| | lin-stimulated | | | |
| | cyclic nucleotide | | | |
| | phosphatase | | | |

TABLE 4
Dot blot analysis of expression of cathepsin B, galectin-1,
RNA-binding protein regulatory subunit and CRBP in
different stages of ovarian cancer and borderline tissues
compared with normal ovary tissues.

| | Tissue types | | |
|---|---|---|---|
| Proteins | and Stage | Signal Strength ^{a)} | Ratio |
| Cathepsin B | Borderline | 373687.2 ± 177135.2 | 2.99 ^{b)} |
| | I + II | 374300.8 ± 108412.8 | 2.99 ^{b)} |
| | III | 378754.6 ± 125816.2 | 3.03 ^{b)} |
| | I˜III | 376775.1 ± 115001.0 | 3.01 ^{b)} |
| | Normal | 125072.3 ± 79980.6 | |
| Galectin-1 | Borderline | 9813.2 ± 2655.1 | 1.04 |
| | I + II | 10276.0 ± 4532.7 | 1.09 |
| | III | 15073.8 ± 4235.9 | 1.60 ^{b)} |
| | I˜III | 12941.4 ± 4896.6 | 1.38 ^{b)} |
| | Normal | 9401.0 ± 4230.3 | |
| RNA-binding | Borderline | 12539.6 ± 2948.0 | 0.63 ^{b)} |
| protein regulatory | I + II | 16127.6 ± 4705.3 | 0.81 ^{b)} |
| subunit | III | 15510.5 ± 3127.5 | 0.78 ^{b)} |
| | I˜III | 15770.4 ± 3760.6 | 0.79 ^{b)} |
| | Normal | 19857.9 ± 4074.0 | |
| Cellular | Borderline | 6828.1 ± 4850.8 | 0.54 ^{b)} |
| Retinol-binding | I + II | 4813.1 ± 3298.0 | 0.38 ^{b)} |
| protein | III | 10771.5 ± 7667.6 | 0.84 |
| (CRBP) | I˜III | 8744.7 ± 6806.3 | 0.69 ^{b)} |
| | Normal | 12748.8 ± 4763.9 | |

^{a)} Each value represents a mean ± S.D. Borderline (n = 10), stage I + II (n = 8), stage III (n = 11), normal ovary (n = 20).
^{b)} P < 0.05, compared with normal.

What is claimed is:

1. A tumor marker for ovarian cancer diagnosis, which is selected from the group consisting of alectin-1, cathepsin B, MHC class I antigen, heat shock protein (HSP) 27, ubiquitin carboxy-termal esterase L1, cellular retinol-binding protein (CRBP), transthyretin, SH3 binding glutamate-rich protein, tubulin-specific chaperone A, RNA binding protein regulatory subunit, γ-actin, tropomyosin and calcium/calmodulin-stimulated cyclic nucleotide phosphatase.

2. A tumor marker as claimed in claim 1, wherein the tumor marker is up-regulated in ovarian tissues from ovarian cancer patients compared with normal ovarian tissues.

3. A tumor marker as claimed in claim 2, wherein the tumor marker is selected from the group consisting of alectin-1, cathepsin B, MHC class I antigen, heat shock protein (HSP) 27, and ubiquitin carboxy-termal esterase L1.

4. A tumor marker as claimed in claim 1, wherein the tumor marker is down-regulated in ovarian tissues from ovarian cancer patients compared with normal ovarian tieeues.

5. A tumor marker as claimed in claim 4, wherein the tumor marker is selected from the group consisting of cellular retinol-binding protein (CRBP), transthyretin, SH3 binding glutamate-rich protein, tubulin-specific chaperone A, RNA binding protein regulatory subunit, γ-actin, tropomyosin and calcium/calmodulin-stimulated cyclic nucleotide phosphatase.

6. A method for detecting ovarian cancer, which comprises the steps of:
(1) obtaining an ovarian tissue sample from an individual to be diagnosed;
(2) determining expression levels of the tumor markers as claimed in claim 1 in the ovarian tissue sample;
(3) comparing the expression levels of the tumor markers in the ovarian tissue sample of step (2) with the expression levels of the tumor markers in non-cancerous ovarian tissues; and
(4) determining if the individual being diagnosed is affected with the ovarian cancer or not from the result of step (3).

7. A method as claimed in claim 6, wherein the expression levels of the tumor markers are analyzed by gel electrophoresis.

8. A method as claimed in claim 6, wherein the expression levels of the tumor markers are analyzed by Western Blot.

9. A method as claimed in claim 6, wherein the expression levels of the tumor markers are analyzed by Dot blot.

10. A method as claimed in claim 7, wherein the non-cancer ovarian tissue is obtained from malignant cells non-invaded region of the same individual to be diagnosed.

11. A method as claimed in claim 6, wherein the ovarian cancer comprises clinical stage I, II, III, and IV ovarian cancer.
